(19) United States
(12) Patent Application Publication
(10) Pub. No.: US 2001/0016765 A1
(43) Pub. Date: Aug. 23, 2001

(54) Method of Identifying Functional Boundaries Between Brain Structures

(75) Inventors: Frans L.H. Gielen, NE Eckelrade (NL); Paul Boulos Bejjani, Adonis, Jounieh (LB)

Correspondence Address: Thomas F. Woods, Medtronic, Inc., MS 301, 710 Medtronic Parkway, Minneapolis, MN, 55432-5604, US

(73) Assignee: Medtronic, Inc.

(21) Appl. No.: 09/818,589

(22) Filed: Mar. 28, 2001

Related U.S. Application Data
(62) Division of application No. 09/394,353, filed on Sep. 13, 1999.
Application No. 09/394,353 is a continuation-in-part of application No. 09/009,247, filed on Jan. 20, 1998, now Pat. No. 6,011,996.

(51) Int. Cl. A61N 1/05

(52) U.S. Cl. 607/116

(57) ABSTRACT

A lead for brain stimulation comprising a macro segment having a macro electrode for test stimulation and subsequent chronic stimulation, and a micro segment having a micro electrode for single cell recording is described. Methods for using the lead to stimulate brain tissue and to identify functional boundaries within brain tissue are also provided.

This application is a continuation-in-part of U.S. application Ser. No. 09/009,247 (Medtronic Docket No. P-7075/MEDT-0106) filed Jan. 20, 1998, entitled “Dual Electrode Lead and Method for Brain Target Localization in Functional Stereotactic Brain Surgery” to Gielen et al., the disclosure of which is hereby incorporated by reference herein in its entirety.

### FIELD OF THE INVENTION

This invention relates to a lead and method for brain stimulation (BS). More particularly, this invention relates to a lead that combines a macro-electrode for test stimulation and subsequent chronic stimulation with a retractable micro-electrode for single cell recording or a semi-micro electrode for multiple cell recording.

### BACKGROUND OF THE INVENTION

Electrical leads are used to stimulate brain tissue in the treatment of such diseases as Parkinson's Disease Tremor and Essential Tremor. One method of brain stimulation is described in U.S. Pat. No. 5,938,688 to Schiff, hereby incorporated herein by reference in its entirety. A typical electrical brain stimulation system comprises a pulse generator operatively connected to the brain by a lead. The lead has one or more stimulating electrodes at its distal end and is designed to be implanted within the patient's brain so that the system of electrodes is optimally and safely positioned for the desired stimulation. U.S. Pat. No. 5,464,446, assigned to Medtronic, Inc. and incorporated herein by reference in its entirety, illustrates a lead anchoring system and discloses a method of positioning the lead so that the electrodes are located at a desired stimulation site. The lead is positioned using a stereotactic instrument which permits precise movements, e.g., +/−1 mm, within the brain.

The initial step towards effective brain stimulation involves localization or mapping of functional brain structures. Especially when the target is new, in the sense that there is little or no statistical data to identify the target location reliably, it is necessary to determine where within the boundary of the functional target area effective and safe stimulation may be delivered.

Therapeutic benefit and non-desired effects of brain lesioning and chronic neuromodulation depend critically on this localization procedure. This procedure involves three primary steps. First, anatomical localization of brain targets is accomplished using anatomical brain atlases, imaging by means of positive contrast x-rays, CT or MRI under stereotactic conditions. Such standard well known imaging techniques are used to make an initial determination of location coordinates for the target to which the lead will be directed.

Second, electrophysiological identification of functional boundaries between brain structures is carried out by means of single- or multi-cell or multi-recording of characteristic cell discharge patterns. Such a procedure may also be referred to as micro recording or semi-micro recording. Micro recording and semi-micro recording require use of an electrode that is small enough to differentiate between single cell activity or multi-cellular activity, and thus requires a micro-electrode with a very small surface area, e.g. between 1-500 square micrometers for a semi-micro-electrode and less than one square micrometer for a micro-electrode.

The third step involves electrical test stimulation within the functional brain structures that have been located. Test stimulation of the selected brain structure is necessary to determine: (1) efficacy of stimulation in the identified functional brain structure, and (2) any side effects caused by stimulation of the brain in this area. If the stimulation electrode is too close to the boundary of the identified brain structure the function of adjacent brain structures may be modulated, which in turn can lead to undesired side effects. Test stimulation is clinically most relevant when performed with an electrode or electrodes having a surface area equivalent to that of the chronic implantable electrodes, e.g., in the range of about 1-20 square millimeters.

Currently, after the first step of determining a target location, a lead containing a micro-electrode is placed in the brain to identify functional boundaries with single-cell recording. Then the lead containing the micro-electrode is withdrawn from the brain tissue. The micro-lead is then replaced with a macro-lead containing a macro-electrode to perform test stimulation. After this step, a further step of withdrawing the macro-lead and replacing it with a third chronic brain stimulation lead may also occur. Those replacements typically require multiple insertions of the leads, all most preferably along the same trajectory path, and therefore increase the risk of intra-cranial hemorrhages with severe permanent disability as a potential consequence. Furthermore, once a lead is positioned and tested to determine that results of stimulation are satisfactory, it is critical that the lead remain in the same place, because even one millimeter of electrode displacement in the wrong direction may cause unsatisfactory results or injury to the brain. Removal of the micro-lead and replacement with the macro-lead also increases the risk that the macro-lead is no longer located in or close enough to the functional target identified by micro recording. Thus it would be desirable to create a lead that is capable of all three functions: single-cell recording, test stimulation and even chronic stimulation.

Theretofore, it has not been possible to perform effective test stimulation with a micro single cell electrode because a micro-electrode is generally insufficient for stimulating a large enough volume of brain tissue to evaluate efficacy and side effects. It has also not been possible to perform effective single cell recording with a macro electrode because a macro electrode senses too large an area for single cell recording. The differences between micro and macro electrode surface areas (typically less than 0.001 square millimeters in comparison to 1 to 20 square millimeters) and their associated current densities, result in stimulation of largely different volumes of brain cells and therefore result in different therapeutic effects and side effects.

Other disclosures relating to the methods and devices for the stimulation of brain tissue include the U.S. Patents listed below in Table 1.

TABLE 1

| U.S. Pat. No. | Title |
|---|---|
| 5,938,688 | Deep brain stimulation method |
| 5,865,842 | System and method for anchoring brain stimulation lead or catheter |
| 5,843,150 | System and method for providing electrical and/or fluid treatment |
| | within a patient's brain |
| 5,843,148 | High resolution brain stimulation lead and method of use |
| 5,833,709 | Method of treating movement disorders by brain stimulation |
| 5,832,932 | Method of treating movement disorders by brain infusion |
| 5,814,014 | Techniques of treating neuro-degenerative disorders by brain infusion |
| 5,800,474 | Method of controlling epilepsy by brain stimulation |
| 5,792,186 | Method and apparatus for treating neuro-degenerative disorders |
| | by electrical brain stimulation |
| 5,752,979 | Method of controlling epilepsy by brain stimulation |
| 5,735,814 | Techniques of treating neuro-degenerative disorders by brain infusion |
| 5,716,377 | Method of treating movement disorders by brain stimulation |
| 5,713,923 | Techniques for treating epilepsy by brain stimulation and drug infusion |
| 5,713,922 | Techniques for adjusting the locus of excitation of neural tissue |
| | in the spinal cord or brain |
| 5,711,316 | Method of treating movement disorders by brain infusion |
| 5,683,422 | Method and apparatus for treating neuro-degenerative disorders |
| | by electrical brain stimulation |
| 5,464,446 | Brain lead anchoring system |
| 5,450,855 | Method and system for modification of condition with neural |
| | biofeedback using left-right brain wave asymmetry |
| 5,402,797 | Apparatus for leading brain wave frequency |
| 5,354,318 | Method and apparatus for monitoring brain hemodynamics |
| 5,331,969 | Equipment for testing or measuring brain activity |
| 5,280,793 | Method and system for treatment of depression with biofeedback |
| | using left-right brain wave asymmetry |
| 5,213,338 | Brain wave-directed amusement device |
| 4,850,359 | Electrical brain-contact devices |
| 4,815,474 | Temporal trajectory analysis in brain electrical activity mapping |
| 4,328,813 | Brain lead anchoring system |
| 4,214,591 | Brain wave analyzing system and method |
| 4,201,224 | Electroencephalographic method and system for the quantitative |
| | description of patient brain states |
| 4,094,307 | Method and apparatus for aiding in the anatomical localization |
| | of dysfunction in a brain |

As those of ordinary skill in the art will appreciate readily upon reading the Summary of the Invention, Detailed Description of the Preferred Embodiments and Claims set forth below, at least some of the devices and methods disclosed in the patents of Table 1 and referenced elsewhere above may be modified advantageously by using the teachings of the present invention.

### SUMMARY OF THE INVENTION

The present invention overcomes at least some of the disadvantages described above by providing a lead for brain stimulation which is capable of micro single cell recording and macro test stimulation. At least one embodiment of the present invention may be implanted for chronic stimulation. The present invention further includes within its scope a lead capable of both micro cell recording and macro test stimulation having an appropriate combination of electrodes having surface areas and configurations appropriate to the volume of brain tissue to be sensed and/or stimulated. Such a combined electrode lead permits delicate single cell recording which is not disturbed by the tissue displacement caused by simultaneous insertion of a macro test stimulation electrode. In one embodiment of the present invention, a micro or semi-micro electrode may be moved or positioned independently of a macro test stimulation electrode attached to the same lead body.

One or more embodiments of the lead of the present invention have certain objects. That is, various embodiments of the present invention provide solutions to one or more problems existing in the prior art, such as: (a) implantable brain stimulation leads capable only of macro stimulation; (b) implantable brain stimulation leads capable only of micro or semi-micro recording; (c) the necessity of creating a trajectory path using a micro or semi-micro lead and then having to remove the lead to perform macro stimulation; (d) the necessity of having to remove a micro recording lead to replace it with a test or chronic stimulation lead; (e) the need for an additional stylet component to a standard deep brain stimulation lead, and (f) the need to use a new micro lead for each new test trajectory.

Various embodiments of the lead of the present invention provide one or more advantages, including: (a) single or multiple cell recording, test stimulation and chronic stimulation with one lead unit; (b) following up single cell recording in one area with test stimulation along the same trajectory; (c) a micro lead capable of performing the function of a supporting stylet, and (d) using a micro lead for more than one trajectory.

Various embodiments of the lead of the present invention have one or more features, including: (a) a lead for brain stimulation that combines micro and macro lead segments and micro and macro electrodes into one unit; (b) a retractable micro segment in a lead; (c) a micro segment in a lead, where the micro segment may be used more than one time, i.e., for making more than one stimulation trajectory path in the brain; (d) a micro segment having appropriate rigidity to support a stylet for a macro electrode or lead; (e) a micro segment which when extended from the lead may be used independently for single cell recording.

Methods of making and using the lead described above also fall within the scope of the invention.

Other features, advantages and objects of the present invention will become more apparent by referring to the appended drawings, detailed description and claims.

### BRIEF DESCRIPTION OF THE DRAWINGS

FIG. 1 is a schematic view of an embodiment of the brain stimulation lead made in accordance with the present invention with the micro segment retracted, held in position by a stereotactic instrument;

FIG. 2 is a perspective view of an embodiment of the brain stimulation lead of the present invention with the micro segment in a extended recording position;

FIG. 3 is a perspective view of the micro segment of FIG. 1;

FIG. 4 is an enlarged view of the recording tip of the micro segment of FIG. 1;

FIG. 5 is a diagrammatic representation of the lead of FIG. 1 relative to a functional portion of the patient's brain; and

FIG. 6 is a flow diagram showing at least some of the steps employed in using the embodiment of the present invention shown in FIG. 1 for making single cell recordings, identifying functional boundaries, carrying out test stimulation and carrying out chronic stimulation.

### DETAILED DESCRIPTION OF THE PRESENTLY PREFERRED EMBODIMENTS

In the specification and claims hereof, the term “lead” is used in its broadest sense and includes within its scope a stimulation lead, a sensing lead, a combination thereof or any other elongated member, such as a catheter, which may usefully be inserted into brain tissue. The term “electrode” means an electrically conductive surface configured for exposure to human tissue and/or fluids and suitable for sensing electrical signals and/or delivering same.

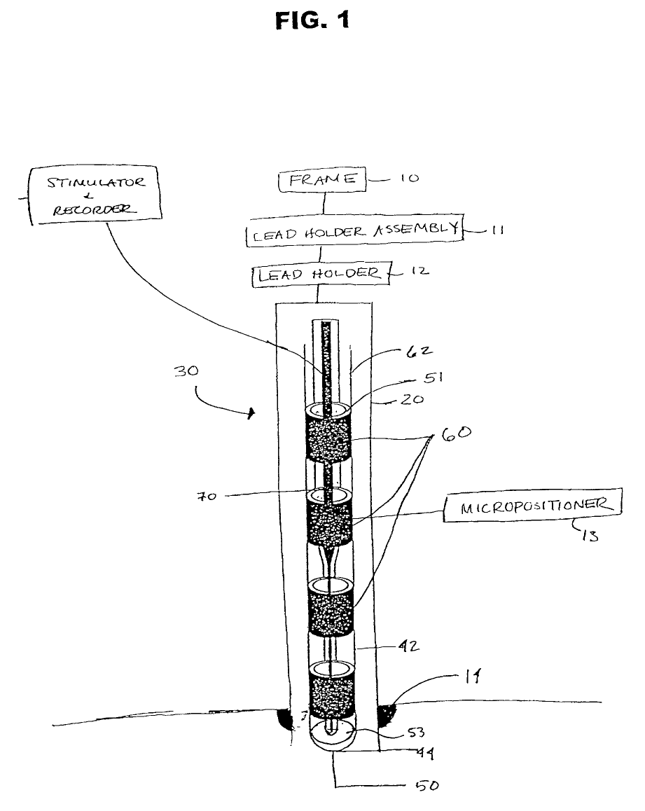

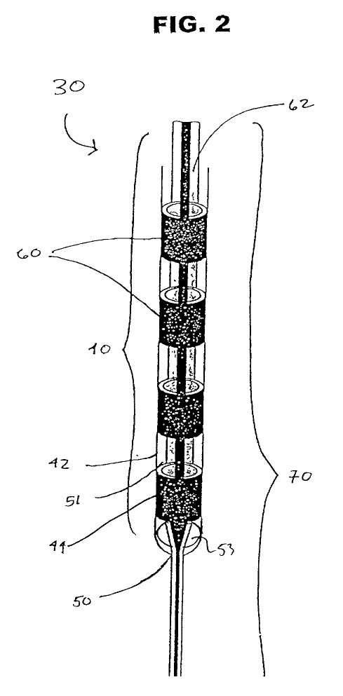

Referring now to FIG. 1 and FIG. 2, a preferred embodiment of a lead of the present invention for brain stimulation is generally shown at 30. Brain stimulation lead 30 comprises macro segment 40 (shown in FIG. 2), including opening 50 forward therein, and micro segment 70 movably positioned within macro segment 40. Micro segment 70 is movable between a retracted position (shown in FIG. 1), where micro segment 70 is housed within macro segment 40, and an extended position (shown in FIG. 2), where micro segment 70 extends through opening 50 of macro segment 40. Optional membrane 53 is configured to permit a stylet or micro segment 70 to be pushed downwardly through a central sealable aperture disposed centrally therein, when segment 70 is pushed downwardly into an extended position. Membrane 53 most preferably prevents the ingress of body fluid into the interior portions of segment 40 when segment 70 is placed in a retracted position at the end of the implant procedure, and also most preferably sealingly engages the side walls of segment 70 when segment 70 is in an extended position during the implant procedure.

As shown in FIG. 1 the stimulation lead may be held in position by a stereotactic instrument. The stereotactic instrument may be a commercially available device and has a frame shown schematically at 10. Frame 10 carries a lead holder assembly indicated at 11, which in turn supports lead holder 12. Lead 30 may be positioned through lead holder 12 and through a guide tube, or cannula, 20. A micro-positioner, shown schematically at 13, may then be used to independently advance brain stimulation lead 30 or micro electrode, semi-micro electrode, or macro stimulation electrode, one cell at a time. The technique for positioning an intra-cranial lead with a stereotactic instrument is well known in the art.

As shown in FIGS. 1 and 2, macro segment 40 most preferably comprises lead casing 42. Macro electrode 44 is preferably positioned at the distal end of casing 42 adjacent opening 50. Macro electrode 44 may have the shape and surface area of a conventional DBS (Deep Brain Stimulation) electrode or BS (Bran Stimulation) electrode, the surface area preferably being in the range of 1 to 20 square millimeters. A system of conductors 60 may extend within casing 42 and preferably extends the length of macro segment 40.

Lead casing 42, made of conventional biocompatible and biostable material such as polyurethane, encapsulates the length of lead 30 in a known manner. Examples of known leads suitable for adaptation for use with the present invention include MEDTRONIC® Model No. 3387 and 3389 DBS leads. A suitable biocompatible material prompts little allergenic response from the patient's body and is resistant to corrosion resulting from implantation within a human body. Furthermore, a suitable biocompatible material should not cause any additional stress to a patient's body. Insulating sleeve 51 retains the coils of conductors 60 and establishes an axial lumen 62 into which a stylet or preferably micro segment 70 may be inserted.

The material from which macro segment 40 is formed may be chosen from a variety of biocompatible, biostable materials. Macro electrode 44 may be made of typical chronic stimulation electrode material such as, e.g., stainless steel, platinum or iridium. Macro segment 40 preferably resembles a typical DBS or BS lead such as the MEDTRONIC Model No. DBS 3280 lead, which is insulated with flexible TEFLON-SILASTIC™ and has platinum/iridium electrodes.

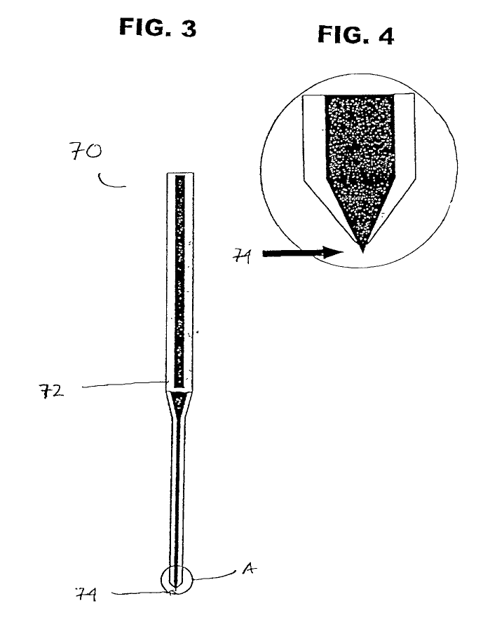

FIG. 1 shows micro segment 70 retracted into opening 50 and within macro segment 40, while FIG. 2 shows micro segment 70 extending through opening 50 and sealable membrane 53. In one embodiment of the present invention, micro segment 70 is left in a retracted position within macro segment 40 after recording and lead 30 is appropriately positioned to enable chronic use. In another embodiment of the present invention and as shown in FIG. 3, micro segment 70 may be completely withdrawn from macro segment 40 and removed from the patient's brain, thus enabling chronic use of lead 30.

Micro-electrode 74 is located at the distal end of micro segment 70 and is preferably positioned at the tip end of micro segment 70. As shown in FIG. 4, micro-electrode 74, at its exposed tip, has an electrode surface area less than about 500 square micrometers, and even more preferably an electrode surface area less than 1 square micrometer for single cell recording applications. For example, electrode 74 may have a surface area of 1 to 500 square micrometers for a semi-micro-electrode, and a surface area of less than 1 square micrometer for a micro-electrode (i.e., 0.1 square micrometers).

Micro segment 70 should be made of material durable for use in making at least one trajectory path, but preferably may be used to make 1 to 10 trajectory paths. The material from which the micro segment is formed may be chosen from a variety of biocompatible, biostable materials such as silicon or tungsten. Micro segment 70 most preferably comprises micro-electrode 74 encapsulated within insulating layer 72. Insulating layer 72 may be made of a variety of biocompatible materials such as polyurethane. Micro-electrode 74 should be made of biocompatible material capable of maintaining strength and rigidity when formed into small diameter objects or shapes. The material currently used in the art which best meets this description is tungsten. Other materials such as platinum or iridium may be used for the micro-electrode 74.

At the beginning of the implant procedure, the distal end of lead 30 is inserted through a burr hole 14 in the skull of the patient. Techniques for positioning an intra-cranial lead with a stereotactic instrument are well known in the art.

The proximal end of lead 30 is connected to an appropriate stimulator and recorder, as illustrated diagrammatically in FIG. 1. Such a stimulator is preferably capable of generating voltage wave trains of any desired form (sine, square wave, spike, rectangular, triangular, ramp, etc.) over a selectable voltage amplitude range (such as from about 0.1 volts to about 10 volts) and over a range of selectable frequencies. In practice, the pulse train and voltage amplitudes employed are selected on a trial and error basis by evaluating a patient's response to various types and amplitudes of electrical stimulation over a course of time ranging between about 1 month to about 12 months.

The length of micro segment 70 when extended (i.e. the length from the distal end of macro segment 40 to the tip of micro segment 70), is most preferably in the range of about 0 mm to about 25 mm, and most preferably ranges between about 1 mm and about 15 mm. Other suitable extension length ranges includes between about 1 mm to about 10 mm, and about 2 mm to about 5 mm. This extension length establishes the distance between the two electrodes 44, 74. This inter-electrode distance is important because larger electrode 44 should be sufficiently remote so that its penetration does not perturb cells being probed by micro-electrode 74. Conductors 60 are coils which connect from the proximal end of the lead 30 to electrodes 44. Micro-electrode 74 is electrically connected through the interior of micro segment 70.

Lead 30 must be stereotactically rigid for insertion into the brain tissue. Current DBS and BS leads contain a stylet to impart the required stereotactic rigidity. In the case of the combined micro-macro lead of the present invention, micro segment 70 may serve the function of giving the required stereotactic rigidity to macro segment 40 and thus to the entire lead 30. Lead 30 must also be of sufficient length to reach the target area of the brain. Since the radius of a typical stereotactic frame is 190 mm, and lead 30 needs to reach the target with a margin of safety, lead 30 is preferably greater than about 30 cm in length.

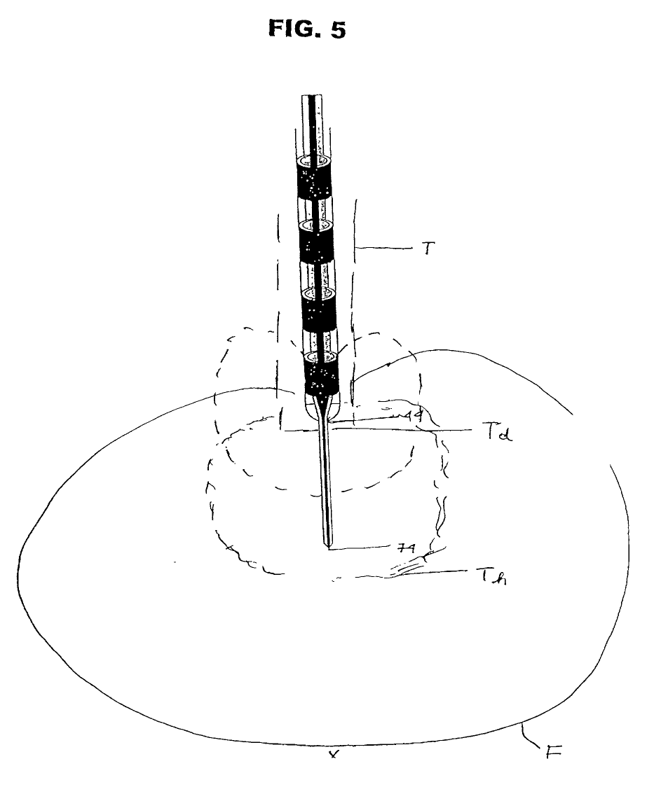

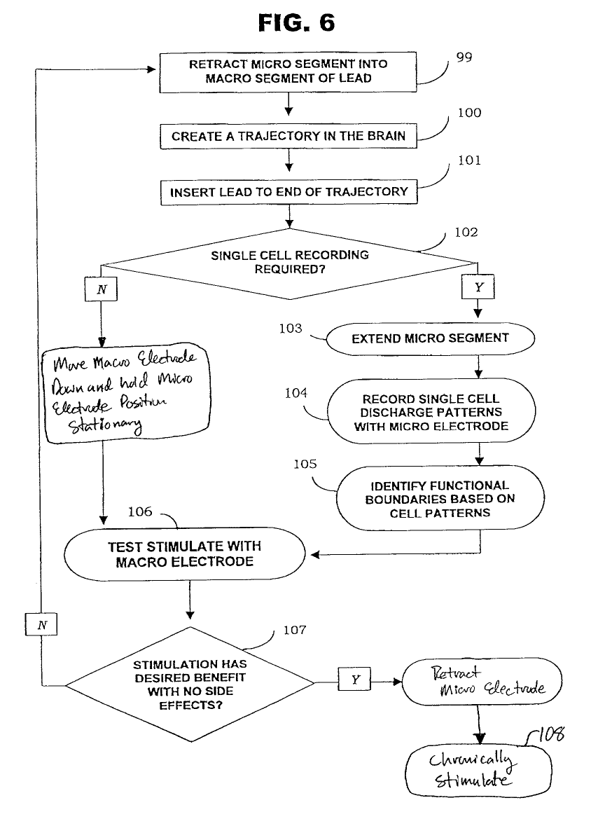

FIG. 5 is a diagrammatic representation of lead 30 relative to a functional portion of the patient's brain, the functional portion being designated by boundary F. Micro segment 70 is shown extended from macro segment 40. Both macro electrode 44 and micro-electrode 74 are shown within functional portion F. The dotted line Tₕ indicates the distance from the macro electrode 44 at which current density delivered by a test stimulus pulse is above the threshold for stimulation.

It is well known that a conventional DBS or BS lead will follow a pre-made stereotactically straight trajectory path inside the brain, such as the trajectory path indicated by T. This trajectory path is generally created by insertion of a stylet-filled hollow tube or cannula. A standard DBS or other BS lead is then inserted through the cannula. Lead 30 of the present invention may also be inserted along such a typically created trajectory path to the deepest part of the pre-made track, designated T_{d}.

The line designated as Tₕ indicates the distance from the macro electrode 44 at which current density delivered by a test stimulus pulse is above the threshold for stimulation. As the tip is moved along trajectory path T indicated by dashed lines through the functional structure cell recordings may be taken using micro-electrode 74 in the extended position and test stimulus pulses may be delivered through macro electrode 44. Knowing the threshold pattern and the distance between the two electrodes, a physician can verify which recorded cells correspond to effective treatment without undesired side effects. It is important to note that in one embodiment of the present invention independent relative movement between the micro electrode or the semi-micro electrode and the macro stimulation electrode is permitted. Such a structure permits micro recording to be accomplished before the brain structure is penetrated with the macro stimulation electrode.

Referring now to FIG. 6, a flow diagram of the primary steps for making single cell recordings, carrying out electrical test stimulation and carrying out chronic stimulation with the lead of the present invention are shown.

Once initial coordinates for the lead target in the brain have been determined using standard imaging techniques, the lead of the present invention may be used according to the method illustrated in FIG. 6. Most preferably, and as indicated at step 99, micro segment 70 is retracted into macro segment 40 of lead 30 before being employed.

As indicated by 100 in FIG. 6, a trajectory path is created such as, for example, trajectory path T in FIG. 5. Such a trajectory path is generally created by insertion of a stylet-filled hollow tube or cannula. A standard DBS or other BS lead is then inserted through the cannula. Lead 30 of the present invention may also be inserted along such a trajectory path to the deepest part of the pre-made track designated T_{d}.

At step 101 micro segment 70 may be in an extended position if lead 30 is employed to perform micro cell recording at the deepest part of the trajectory path T_{d}, which is generally near boundary F of the functional structure. More preferably, however, at step 101 of FIG. 6 micro segment 70 is in a retracted position and lead 30 is configured such that macro electrode 44 is positioned at the deepest part of trajectory path T. If, for example, the functional structure is well known, the surgeon may elect to begin test stimulation at step 102, thereby proceeding to step 106.

If, however, the functional structure is not well known, at step 102 the surgeon may elect to begin the procedure with single cell recording to determine functional boundaries, thus proceeding to steps 103 and 104 in which micro segment 70 is extended a single cell at a time and cell discharge patterns are recorded with micro electrode 74. Those patterns are used at step 105 to identify functional boundaries.

In FIG. 5, steps 103 through 105 occur as micro electrode 74 advances a single cell at a time from T_{d} to Tₕ. After step 105, a test stimulation pulse is delivered (step 106), and it is determined whether stimulation is efficacious and does not cause undesired side effects in the area where the test stimulation has been delivered. Single cell recording (steps 103 through 105) followed by test stimulation 106 can be repeated in such a manner until micro electrode 74 has passed through or out of the functional structure area. In FIG. 5, for example, once micro electrode 74 reaches point X, micro electrode 74 is no longer disposed in the functional area.

Once lead 30 has completed its travel through the functional area (i.e., lead 30 has been positioned beyond functional boundary F), the efficacy of the stimulation is determined as indicated at step 107 in FIG. 6. If the test stimulations of step 106 prove unsatisfactory, a new trajectory path may be established and steps 99 through 106 may be repeated until a suitable stimulus location has been found.

As indicated above, in any given case the establishment of a number of different trajectory paths may be required to find a suitable stimulus location. Micro segment 70 is, therefore, preferably formed of a material that may be employed to form more than one trajectory path.

Following the determination of a suitable location for chronic stimulation, the entire combined micro-macro lead 30 of the present invention may be withdrawn and replaced with a standard chronic deep brain stimulation lead. It is preferred, however, that micro segment 70 be withdrawn and macro segment 40 remain in a fixed location with respect to the patient's skull. Chronic stimulation may then be carried out as shown at step 106, preferably by generating and delivering stimulus pulses through the macro segment 40 and macro electrode 44. Accordingly, the lead of this invention may still be used as a test lead which is disposed of after determining a chronic stimulation site or it may preferably be used both for the test procedure and chronic stimulation. It is important to note that the scope and application of the present application are not limited to DBS applications, devices, and methods but extend to devices and methods for sensing and stimulating other regions or portions of the brain.

Although specific embodiments of the invention have been set forth herein in some detail, it is to be understood that this has been done for the purposes of illustration only, and is not to be taken as a limitation on the scope of the invention as defined in the appended claims. It is to be understood that various alternatives, substitutions and modifications may be made to the embodiment describe herein without departing from the spirit and scope of the appended claims.

In the claims, means-plus-function clauses are intended to cover the structures described herein as performing the recited function and not only structural equivalents but also equivalent structures. Thus, although surgical glue and a screw may not be structurally similar in that surgical glue employs chemical bonds to fasten biocompatible components together, whereas a screw employs a helical surface, in the environment of fastening means, surgical glue and a screw are equivalent structures.

All patents cited hereinabove are hereby incorporated by reference into the specification hereof, each in its respective entirety.

We claim:

1. A brain stimulation lead, comprising:
a macro segment having an opening disposed therein; and
a micro segment movably positioned within the macro segment, the micro segment being movable between a retracted position when the micro segment is housed within the macro segment and an extended position when the micro segment extends through the opening of the macro segment.

2. The lead of claim 1, wherein the macro segment further comprises at least one electrode adjacent the opening.

3. The lead of claim 2 wherein, the electrode is configured to perform test stimulation of brain tissue.

4. The lead of claim 2 wherein, the electrode has a surface area ranging between about 1 mm² and about 20 mm².

5. The lead of claim 1, wherein the micro segment further has a distal end and a proximal end and comprises at least one micro-electrode disposed near the distal end, the micro-electrode being configured to perform single cell recording of brain tissue.

6. The lead of claim 2, wherein the micro segment further includes a distal end and a proximal end and comprises at least one micro-electrode disposed near the distal end, the micro-electrode being configured to perform single cell recording of brain tissue.

7. The lead of claim 5, wherein the micro-electrode has a surface area ranging between about 1 μm² and 500 μm².

8. The lead of claim 5, wherein the micro-electrode has a surface area of less than about 1 μm².

9. The lead of claim 5, wherein the micro-electrode further comprises tungsten.

10. The lead of claim 1, wherein the length of the micro segment in the extended position ranges between about 1 mm and about 10 mm.

11. The lead of claim 1, wherein the macro segment is configured to follow a straight trajectory path in the brain and the micro segment is configure to follow the same trajectory path.

12. The lead of claim 1, wherein the micro segment comprises material suitable for use in making a plurality of trajectory paths in human brain tissue to be used in a plurality of trajectories.

13. A method of stimulating brain tissue, comprising:
providing a lead comprising a macro segment including an opening disposed therein and a macro electrode adjacent the opening, a micro being segment movably positioned within the macro segment;
retracting the micro segment into the opening in the macro segment;
establishing a trajectory path within brain tissue;
inserting the lead along the trajectory path while the micro segment is in a retracted position; and
stimulating the brain tissue with the macro electrode.

14. The method of claim 13, further comprising:
providing a micro electrode positioned at a distal end of the micro segment;
extending the micro segment through the opening;
recording cell discharge patterns with the micro-electrode;
identifying functional boundaries based on the cell discharge patterns; and
stimulating the brain tissue with the macro electrode within the functional boundaries.

15. A method of identifying functional boundaries between brain structures, comprising:
providing a lead comprising a macro segment including an opening disposed therein and a macro electrode adjacent the opening, a micro segment being movably positioned within the macro segment, the micro segment comprising a micro-electrode disposed at a distal end thereof;
retracting the micro segment into the opening;
establishing a trajectory path within brain tissue;
inserting the lead along the trajectory path while the micro segment is retracted;
extending the micro segment through the opening;
recording cell discharge patterns with the micro-electrode; and
identifying functional boundaries based on the cell patterns.

16. A brain stimulation system, comprising:
a lead comprising a macro segment having an opening disposed therein; a micro segment movably positioned within the macro segment, the micro segment being movable between a retracted position when the micro segment is housed within the macro segment and an extended position when the micro segment extends through the opening of the macro segment;
at least one electrode adjacent the opening of the macro segment configured to permit test stimulation of brain tissue; and
at least one electrode disposed near a distal end of the micro segment configured to permit single cell recording.

17. The lead of claim 16, wherein the macro segment is operatively adapted to follow a straight trajectory in the brain and the micro segment is operatively adapted to follow the same trajectory.

18. The lead of claim 16, wherein the micro segment is made of material suitable to be used in a plurality of trajectories.

19. A method of stimulating brain tissue, comprising:
retracting a micro segment into a macro segment;
creating a trajectory path within brain tissue;
inserting a macro segment along the trajectory path while the micro segment is in a retracted position;
extending the micro segment;
recording cell discharge patterns with the micro-electrode;
identifying functional boundaries based on the cell discharge patterns; and
stimulating the brain tissue with the macro electrode within the functional boundaries.

20. A brain stimulation lead, comprising:
identifying means for determining functional boundaries of brain tissue;
means for performing test stimulation of brain tissue; and
means for removing the identifying means from a brain tissue site during test stimulation.

21. The lead of claim 20, further comprising:
means for creating a trajectory path within brain tissue.

22. The lead of claim 20, further comprising means for chronic stimulation of brain tissue.

23. A brain stimulation lead comprising:
identifying means for determining functional boundaries of brain tissue;
means for performing test stimulation of brain tissue; and
means for housing the identifying means.

24. A system for brain stimulation comprising:
identifying means for determining functional boundaries of brain tissue;
means for performing test stimulation of brain tissue;
means for housing the identifying means; and
means for removing the identifying means from a brain tissue site during test stimulation.
